(19) United States
(12) Patent Application Publication
(10) Pub. No.: US 2015/0209706 A1
(43) Pub. Date: Jul. 30, 2015

(54) Lid for liquid filtration

(71) Applicant: John Milner McCary, SR., Birmingham, AL (US)

(72) Inventor: John Milner McCary, SR., Birmingham, AL (US)

(21) Appl. No.: 13/999,197

(22) Filed: Jan. 28, 2014

(51) Int. Cl. B01D 35/02; A61M 3/02

(52) U.S. Cl. CPC B01D 35/02; A61M 3/0241; A61M 2210/0618

(57) ABSTRACT

The proposed invention is a tilt-to-pour personal consumed liquid filtering lid that attaches to the top of liquid containers, such as cups, bottles and mugs where the consumer tilts the container over to drink or pour out the lid edge outlet or neti pot style spout outlet. A liquid filter holding section that holds a liquid filter is attached underneath the tilt-to-pour filter lid. The tilt filter lid is manufactured such that the filter holding section liquid inlet is oriented toward the tilt filter liquid outlet. When the liquid container is tilted over, toward the tilt filter lid liquid outlet side, liquid flows from the liquid container into and through the liquid filter inlet, then flows into the liquid filter holding section through the liquid filter and then the liquid flows out through the liquid outlet.

### FIELD OF THE INVENTION

The present invention relates to the liquid filtration method and means of a liquid filtering lid placed on a liquid dispensing container.

### BACKGROUND OF THE INVENTION

There are many different styles of personal individual consumed water or liquid filter containers available in the market and patented that include plastic and/or metal containers or bottles with various styles of water filters. The personal water or liquid filters either go down inside the water or liquid container or are part of the cap system of the water/liquid filtering container. There is some separate lid or cap water or liquid filters that can be attached, usually screwed on, to various bottles. In most all of these personal water filtering dispensers the water or liquid is drawn out either through a straw type device; sucked out; squeezed out the top center of the container or partially gravity fed out the top center of the personal water or liquid filtering container. There is not a personal individual consumed tilt-to-pour gravity flow liquid filtering lid patented or on the market.

### SUMMARY OF THE INVENTION

The present invention is a tilt-to-pour snap-on; screw-on and/or multi-size stretch and snap over personal consumed or nasal flushing liquid filtering lid. Tilt-to-pour lids are those lids that are usually snapped on to the lip of plastic cups or mugs, such as the Tervis brand, and also seen as lids placed onto plastic or paper coffee cups at fast food restaurants or convenience stores. The liquid consumer tilts the liquid container with lid over and the consumer drinks or pours from the lid outlet at or near the edge of the lid, with the majority of the liquid gravity flowing out the lid outlet. These same tilt-to-pour lids can be placed on soft drink dispenser cups. Childs “sippy cups” is another example of a tilt-to-drink liquid dispenser. Another application is where the liquid might not be directly consumed but rather personal nasal poured such as in a “neti pot” style application where there is the need for a one micron or smaller filter (NSF 53 or 58) to filter out harmful bacteria and germs that may infect the nasal lining.

This personal water or liquid tilt-to pour filtering lid (tilt filter lid) can be placed on any cup, tumbler, mug or container that the lid is sized and has the correct dimensions to fit snugly onto the lip of the container. In patent U.S. Pat. No. 8,464,633 and Application US 2008/0067120 both of the filtering caps are center suction or squeeze type filtering bottle or container caps and are not tilt-to-pour style lids. The lid top of the tilt filter lid in many instances has a slide stopper device to block the containers liquid from coming out the top liquid outlet.

The design of the tilt-to-pour filter system and liquid delivery to the user is different than other personal top center liquid filtering dispensers. The filter system of the tilt-to-pour filter lid needs to be specifically configured so that when the tilt filter lid is placed onto the tilt-to-pour container the unfiltered liquid that comes from the container can flow efficiently and effectively into the liquid filtering section, then out the tilt-to-pour filter outlet that is near or on the top edge of the lid. As the container is tilted over and the user drinks or nasal pours the liquid, the majority of the liquid flowing out the lid outlet or nasal spout is gravity fed. The inlet of the underneath filter lid filtering section is placed close to the “sipping” drinking or nasal pouring side of the container so that the unfiltered container liquid flows into the filter section when the tilt filter container is tilted over by the user. This tilt over liquid catching filter inlet configuration is especially effective when the liquid in the tilt filter is low and there is the need to get most of the final liquid into and through the filter and out the tilt filter outlet to the tilt-to-pour lid user. When drinking the tilt-to-pour filter lid container user will in most case not need to apply much, if any, sucking of the containers liquid out the tilt filter lid outlet to have the tilt filter lid liquid flow in a comfortable mostly gravity fed liquid delivery.

One embodiment of the present invention is to have semi disposable tilt-to-pour filter lids that are used at fast food or convenience stores. The tilt filter lid may be given to or sold to the user. The tilt-to-pour lid filter user can then get tap water or other liquid in the provided container and then places the tilt-to-pour filter lid on to the liquid container. The use of the tilt-to-pour filter lid would be very useful in geographies where the drinking water may be considered unpleasant or possibly harmful to drink.

Another embodiment of the present invention is to have an additive dispenser added to the manufactured tilt-to-pour lid assembly. The additive dispenser is usually designed where the additive is added to the liquid after the liquid has been filtered, but not yet poured by the tilt filter lid user. The tilt filter lid additive may be in a tablet, powder or possibly liquid form and the additive filter may have a dispensing control screen or sieve.

Still, another embodiment is to have the tilt-to-pour filter lid placed upon a neti pot container that is used to flush out nasal sinuses through a pouring spout that is at or near the top of the neti pot container. The filter, usually a one micron or less filtering mechanism, is placed between the flushing container liquid and the outflow entrance of the spout where the flushing liquid is filtered as the filter-to-pour lid covered neti pot container is tilted over and the flushing fluid flows out of the spout outlet.

### BRIEF DESCRIPTION OF THE INVENTION

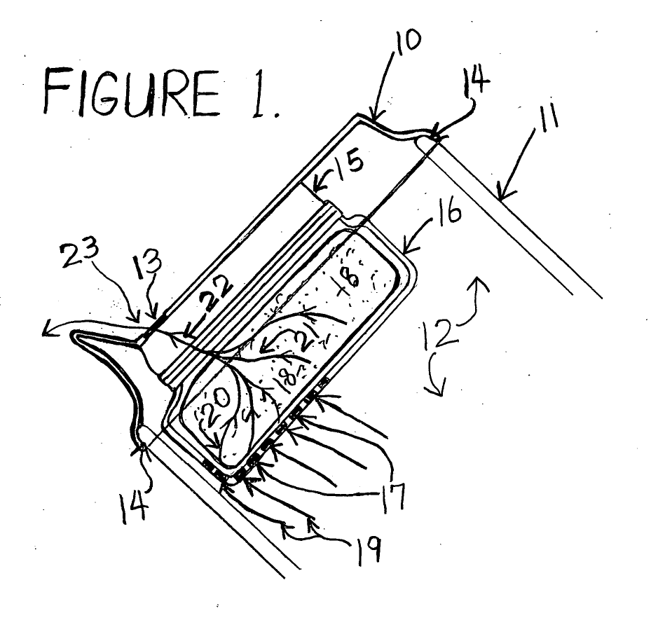

FIG. 1 in a side perspective view is the tilt-to-drink filtering lid placed upon a liquid container tilted over.

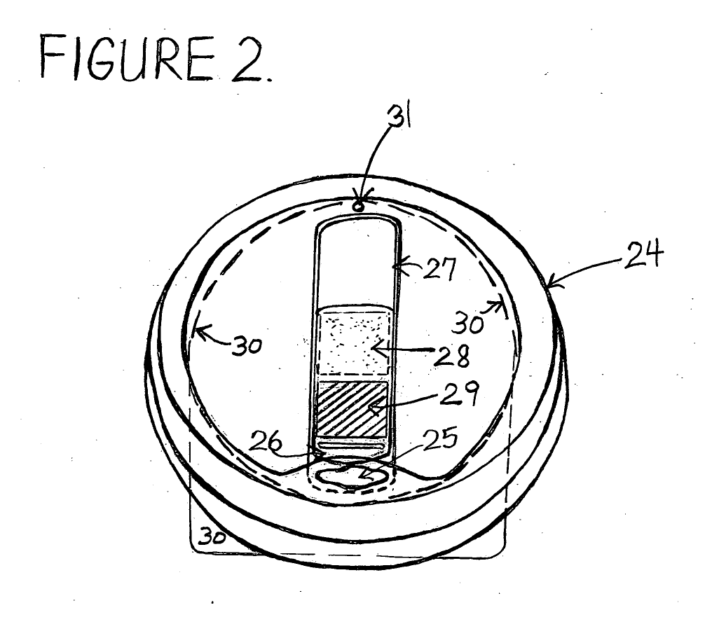

FIG. 2, in an overhead perspective view, is the additive dispenser on the tilt filter lid and the lid outlet closer cover.

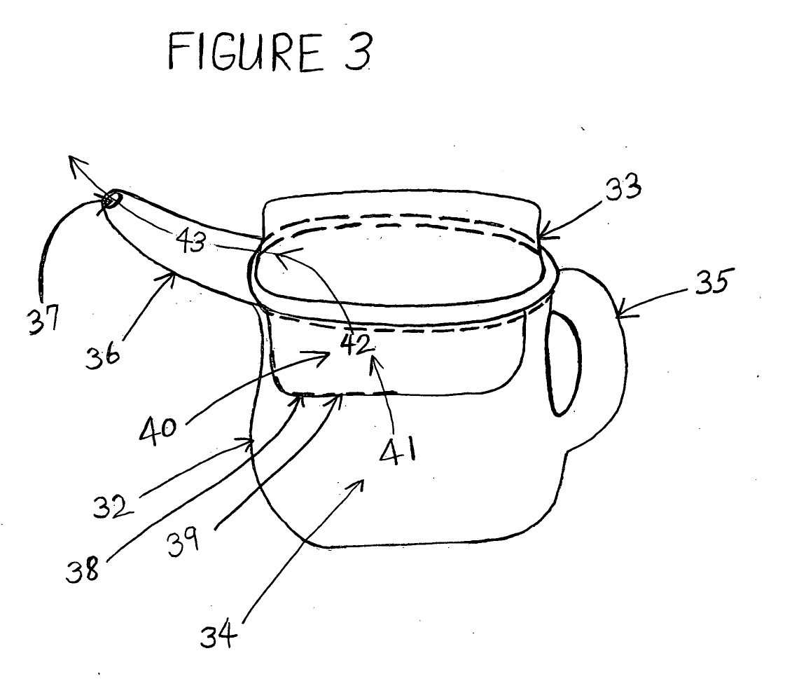

Shown in FIG. 3, in a side perspective view, is the application of the tilt filter lid being used in a neti pot nasal flushing application.

### DETAILED DESCRIPTION OF THE INVENTION

Shown in FIG. 1, in a side perspective view, is the tilt-to-drink filter lid 10 placed on a liquid container 11 that holds various forms of liquid 12. The tilt filter lid outlet 13 and lid snap over 14 attaching the tilt filter lid to the liquid container 11. The tilt filter lid filter holding section attaching point 15 is shown attached underneath the lid with filter holding section 16 attached. The filter holding sections inlets are 17 are show with the liquid filter 18 placed in the filter holding section 16. As the liquid container is tilted over the container liquid 12 flows into 19 the filter holding section inlets 17 into 20 and through 21 the filter 18 and through 22 the tilt filter lid outlet 13 and out 23 the tilt filter lid 10.

FIG. 2 shows in an overhead perspective view the tilt filter lid 24 with the lid outlet 25. The lid closer 26 is shown along with the lid closer guide 27, the additive dispensing slot 28 and the dispensing slot lid closer opening 29 that allows access to the dispensing slot 28. The liquid filter assembly 30 is shown attached underneath the lid and the air inlet 31 is also shown on the top of the lid.

Shown in FIG. 3 in a side perspective view is the tilt filter lid as part of a neti pot style nasal flushing container 32. The tilt filter lid 33 is shown attached on top of the neti pot container 32 which contains the nasal flushing liquid 34. Also shown is the neti pot container handle 35, the spout 36 and the spout hole 37. The filter inlet holes 38 are shown on the lower surface of the filter container 39 which contains the one or less micron filter 40. The neti pot flushing liquid 34 flows 41 through the filter 42 through and out the spout 43.

1. A manufactured assembly of a tilt-to-pour direct gravity flow through liquid filtering lid that is attached on to a liquid container, comprising:
a. a tilt-to-pour filter lid, wherein the tilt-to-pour filter assembly has a lid that can be firmly attached to the top of a liquid container, which container includes a cup, mug or bottle; and
b. a liquid outlet, wherein the tilt-to-pour filter lid has a liquid outlet on or near the top edge surface of the lid; and
a liquid filter holding section, wherein the tilt-to-pour lid filter has a liquid filter holding section attached underneath on the bottom surface of the tilt-to-pour filter lid and the filter holding section is significantly oriented toward the liquid filter outlet in that the filter holding section does not cover the entire space underneath the filter lid; and
filter inlets, wherein the liquid filter holding section has inlets that liquid from the liquid container flows through and into the liquid filter holding section
f. a liquid filter, wherein liquid filter material is placed in the liquid filter holding section, and the filter holding section are manufactured such that where the filter holding section is located toward the tilt filter lid liquid outlet side of the lid such that, the manufactured tilt-to-pour filtering lid assembly allows that when the liquid container is tilted over toward the tilt-to-pour filter liquid outlet side of the attached lid, liquid in the lower section of the liquid container gravity flows from the liquid container into and through the liquid filter inlets, the liquid flows through liquid inlets flowing into the liquid filter holding section, flowing through and saturating the liquid filter located in the liquid filter holding section, the filtered liquid flows past the liquid filter holding section out through the tilt-to-pour filter outlet.

2. A manufactured assembly of a tilt-to-pour direct gravity flow through liquid filtering lid that is attached on to a liquid container according to claim 1, wherein an air inlet is on the top surface of the tilt-to-pour filter lid.

3. A manufactured assembly of a tilt-to-pour direct gravity flow through liquid filtering lid that is attached on to a liquid container according to claim 1, wherein an additive dispensing section is placed on and into the top surface of the tilt-to-pour filter lid.

4. (canceled)

5. A manufactured assembly of a tilt-to-pour gravity flow through liquid filtering lid that is attached on to a liquid container according to claim 1, where the tilt filter lid can be stretched attached to snap on and over the top lip of various size liquid containers.

6. (canceled)

7. A manufactured assembly of a tilt-to-pour direct gravity flow through liquid filter lid that is attached on to a liquid container according to claim 1, wherein the filter held by the filter holding section can be replaceable.

8. A manufactured assembly of a tilt-to-pour direct gravity flow through liquid filter lid that is attached on to a liquid container according to claim 1, wherein the bottom part of the filter holding section is open allowing the gravity flow liquid to flow from the liquid container directly through the filter that is held in place by the filter holding section and the filter holding section inlets may not be present.
